(19) United States
(12) Patent Application Publication
(10) Pub. No.: US 2020/0078274 A1
(43) Pub. Date: Mar. 12, 2020

(54) GELLAN SEAMLESS BREAKABLE CAPSULE AND PROCESS FOR MANUFACTURING THEREOF

(71) Applicant: V MANE FILS, Bar Sur Loup (FR)

(72) Inventors: Jean-Michel HANNETEL, GRASSE (FR); Didier HARTMAN, CANNES LA BOCCA (FR); Nathalie COURSIERES, SAINT CEZAIRE SUR SIAGNE (FR); Jean MANE, GRASSE (FR)

(21) Appl. No.: 16/575,865

(22) Filed: Sep. 19, 2019

Related U.S. Application Data
(63) Continuation of application No. 11/922,574, filed on Feb. 1, 2008.

(30) Foreign Application Priority Data
Jun. 21, 2005 (EP) PCT/EP2005/008502
Aug. 5, 2005 (EP) PCT/EP2005/009226

(51) Int. Cl. A61K 8/11; A61K 9/48; A61Q 11/00; B01J 13/04; A61Q 19/00; A23L 29/269; A23P 10/30; A23L 27/00

(52) U.S. Cl. CPC A61K 8/11; A61K 9/4833; A61Q 11/00; A61K 9/4816; B01J 13/046; A23V 2002/00; A23L 29/272; A23P 10/30; A23L 27/72; Y10T 428/2991; A61K 9/4858; A61Q 19/00

(57) ABSTRACT

The invention relates to a process for manufacturing a seamless breakable capsule, comprising —co-extruding an external and hydrophilic liquid phase, and an internal and lipophilic liquid phase, in order to form a capsule constituted of a core comprising the internal and lipophilic phase, and a shell comprising the external and hydrophilic phase, —immersing into an aqueous solution containing a curing agent, wherein the external liquid phase includes a gelling agent comprising gellan gum alone or in combination with another gelling agent, a filler, and a divalent metal sequestering agent, and to breakable capsules comprising a core and a shell, wherein the shell includes a gelling agent comprising gellan gum alone or in combination with another gelling agent, a filler, and a divalent metal sequestering agent.

### BACKGROUND OF THE INVENTION

### Field of the Invention

The present invention relates to a breakable capsule having a fluid core and a solid breakable shell.

In this invention, the term “capsule” means a delivery system of a substance, said substance being hereinafter referred to as “the core”, which is enclosed into a shell. The term “breakable capsule” refers to a capsule as hereabove defined, wherein the shell can be ruptured by means of a pressure, which results in the release of the core.

Such capsules are useful for numerous applications, such as in oral care application (toothpaste, mouthwash, gums . . . ), in food applications such as confectionary, dairy, bakery, savory, or in personal care products such as cosmetic products and the like.

In the present patent application, the term “capsule” will be used to designate any size of capsules, including macrocapsules and microcapsules and preferably capsule from 0.5 up to 8 mm.

It is of particular interest to obtain seamless capsules, as the breakability of a welded capsule may be influenced by the easy rupture of the weld.

### Description of the Related Art

Fuji patent application JP10291928 describes a capsule obtained through a co-extrusion process, wherein the external liquid phase comprises gellan and calcium salts. Gellan gum, first discovered in 1978, is produced by the microorganism Sphingomonas elodea.

The Applicant has found that the production of gellan capsule through the Fuji process was not satisfactory and resulted in poor quality capsules and in processing difficulties, because the gellan was actually gelling during the co-extrusion, and it was not possible to obtain spherical and homogeneous breakable capsules.

For this reason, the Applicant tried to improve the Fuji process and found that the drawbacks of the prior art process may be due to the presence of calcium salts, and more generally to divalent metal salts in gellan during the co-extrusion step. Thus, the Applicant experimented a process wherein the co-extrusion liquid phase containing gellan was performed in absence of calcium salts, and observed that, surprisingly, the resulting capsules had the required spherical shape and homogeneous size. However, the obtained capsules cannot be used as such, because the shell is too soft and the resulting capsules are not breakable capsules; the Applicant found a solution to this subsequent technical problem by contacting the capsules with divalent metal ions, preferably calcium or magnesium ions, once the co-extrusion process is finished, and this finally lead to satisfactory breakable capsules.

### SUMMARY OF THE INVENTION

Thus, this invention relates to a process for manufacturing seamless breakable capsules and to new breakable capsules.

The process of the invention comprises a step (A) of co-extrusion of an external and hydrophilic liquid phase and an internal and lipophilic liquid phase, in order to form a capsule having a core comprising the internal and lipophilic phase and a shell comprising the external and hydrophilic phase; and a step (B) of washing and immersing the capsules into an aqueous solution containing a curing agent, suitable for making the shell breakable as required for the intended use; optionally a step (C) of drying the obtained capsules in a dry air or optionally a step (D) of suspending the capsules into an aqueous medium to obtain a slurry form.

The co-extrusion process consists of three main stages: compound drop formation, shell solidification and capsule collection. The compound drop is a sphere of the liquid fill phase inside the shell phase. The liquid fill phase is hereinafter referred to as “the core”. The shell phase is hereinafter referred to as “the shell”.

According to the invention, the external liquid phase includes a gelling agent comprising gellan gum alone or in combination with another gelling agent, a filler, and a metal sequestering agent, the liquid being water, preferably desionized or osmozed water.

By “gelling agent” in the meaning of this invention, it is referred to an agent able to convert an aqueous phase from a flowable liquid to a solid or a gel.

By “sequestering agent” in the meaning of this invention it is referred to any agent complexing, chelating or sequestering bivalent ions such as calcium or magnesium.

The breakable capsule according to the invention is characterized in that it has a crush strength is comprised between 0.01 and 5 kp, preferably 0.1 to 2.5 kp. The crush strength of the capsule is measured by continuously applying a load vertically onto one particle until rupture. The crush strength of the capsules in the present invention is measured by using a texturometer TA.XT plus from Micro Stable System in compression mode or a LLOYD-CHATILLON Digital Force Gauge, Model DFIS 50, having a capacity of 25 Kg, a resolution of 0.02 Kg, and an accuracy of +/−0.15%. The force gauge is attached to a stand; the capsule is positioned in the middle of a plate that is moved up with a manual thread screw device. Pressure is then applied manually and the gauge records the maximum force applied at the very moment of the rupture of the capsule, (measured in Kg or in Lb). Rupture of the capsule results in the release of the core.

Gellan gum is a hydrocolloid which, according to the invention, can be used as the sole gelling agent of the external liquid phase, or in combination with other gelling agents. Other suitable gelling agents may be alginates, agar, carragheenan, xanthan gum, dextran, curdlan, welan gum, rhamsan gum or modified starches. Suitable gellan gums are for example, but not limited to deacylated gellan gum. Kelcogel® can be mentioned as a suitable gellan gum.

The amount of gelling agent present in the shell is 4 to 95%, preferably 5 to 75%, even more preferably is 10 to 50% by weight of the total dry weight of the shell.

When used in combination with at least another gelling agent, the weight ratio between gellan gum and the other gelling agent(s) is from 80/20 to 20/80, preferably 75/25 to 25/75, and even more preferably from 60/40 to 50/50.

The filler is any suitable material that can increase the percentage of dry material in the external liquid phase and thus after co-extrusion in the obtained shell. Increasing the dry material amount in a shell results in solidifying the shell, and in making it physically more resistant. Preferably, the filler is selected from the group comprising starch derivatives such as dextrin, maltodextrin, cyclodextrin (alpha, beta or gamma), or cellulose derivatives such as hydroxypropylmethylcellulose (HPMC), hydroxypropylcellulose (HPC), methylcellulose (MC), carboxymethylcellulose (CMC), polyvinyl alcohol, polyols or mixture thereof.

The amount of filler in the shell is at most 98.5%, preferably from 25 to 95% and even more preferably from 50 to 80% by weight on the total dry weight of the shell.

Using a divalent metal sequestering or complexing agent allows to trap the divalent metal ions which are possibly present in the components of the liquid phase including water and which have a gelling effect on gellan. Thus, the use of a divalent metal sequestering agent, preferably of a calcium ion sequestering agent, allows the gellan to be co-extruded without undesirable or uncontrollable gelling during the coextrusion.

The amount of sequestering agent is at most 2%, preferably at most 1% and even more preferably at most 0.5% by weight of the total dry weight of the shell.

Preferably, the water used for the external phase is deionized water or osmozed water; using processing water remains possible but needs adjusting the amount of divalent metal sequestering agent.

The sequestering agent is a metal salt, preferably selected from the group comprising trisodium citrate, trisodium phosphate, tetrasodium pyrophosphate, sodium hexametaphosphate and mixtures thereof.

The hydrophilic external liquid phase may further comprise at least one plasticizer, which may be glycerol, sorbitol, maltitol, triacetine or polyethylene glycol type, or another polyalcohol with plasticizing or humectant properties, and advantageously a coloring agent or pigment in a form of powder or suspension stable in aqueous medium.

According to one embodiment of the invention, the co-extrusion step (A) of the process can be performed at a temperature being from room temperature to 100° C. Advantageously, it is performed at room temperature, which means between 18 and 30° C., preferably 20-25° C. under atmospheric pressure.

The co-extrusion step is a synchronous extrusion of two liquids: the external and hydrophilic liquid phase, and the internal and lipophilic liquid phase which can be performed using an apparatus and a process as described in EP 513603, the disclosure of which is herein incorporated by reference.

According to an embodiment of the invention, after the co-extrusion step (A), the solidification step is performed by keeping cold the capsules in order to ensure correct gelling of the shell, for example by contacting them with a cold bath. The cold bath may preferably be cold oil or cold emulsion. The capsules may then be centrifuged in order to remove the surplus oil, and/or dried and washed with organic solvent (such as acetone, ethyl acetate, ethanol, petroleum ether, etc.) also to remove the surplus oil, and optionally dried in a current or air at controlled temperature and humidity. The relative humidity of the drying air is 20% to 60%, preferably 30 to 50%; the temperature of the drying air is of 15 to 60° C., preferably 35 to 45° C.

The thus obtained capsules are then immersed into an aqueous solution or an emulsion containing a curing agent which comprises a divalent salt and optionally an acid. The effect of the immersion step is to wash out the oil remaining at the periphery of the capsule, and to gradually strengthen the shell, notably through dehydration and osmotic equilibrium.

According to one embodiment of the invention, after immersion, the capsules are dried in the same conditions as mentioned above.

The curing agent preferably comprises divalent metal ions, or a mixture of divalent metal ions, such as calcium ions or magnesium ions.

The aqueous solution containing the curing agent is preferably a divalent metal salt solution, preferably containing calcium or magnesium salts, more preferably, calcium dichloride, calcium carbonate, calcium sulfate or dicalcium phosphate. This solution may be the aqueous phase of an oil-in-water emulsion. This solution can be at a temperature comprised between 2° C. and room temperature. Advantageously, the aqueous solution containing the curing agent is maintained under acid conditions of pH, and preferably at a pH less than 5, more preferably from 3 to 4. According to a preferred embodiment of the invention, the aqueous solution containing a curing agent is a calcium chloride solution having a pH of 3 to 4.

The aqueous solution containing the curing agent can also contain preservatives or bactericides such as benzoate, parabens, diols, cetylpyridinium chloride, diazolidinyl urea or any preservatives used for food, pharmaceutical or cosmetic products.

According to one embodiment of the invention, the process comprises the steps of co-extruding the above mentioned external and internal liquid phases, optionally solidifying and/or gelling the surface of the shell by keeping the capsule under cold conditions, optionally centrifugating, optionally washing the so-obtained capsules with an organic solvent, immersing the resulting capsules into an aqueous solution containing a curing agent, and drying the capsules.

According to one embodiment of the invention, the solidifying/gelling/curing steps can be gathered into a single step, for example by dipping the capsules into a bath, under cold conditions, containing the divalent metal salts, preferably calcium or magnesium salts, more preferably, calcium dichloride, calcium sulfate or dicalcium phosphate. This bath may be an oil-in-water emulsion.

The capsules manufactured through the process according to the invention are essentially or perfectly spherical and very homogeneous in size.

This invention also relates to breakable capsules which are preferably seamless capsules susceptible to be obtained through the process according to the invention.

The capsule of the invention comprises a core and a shell, and said shell includes a gelling agent comprising gellan gum alone or in combination with another gelling agent, a filler, and a divalent metal sequestering agent.

Preferably the gelling agent of the shell is a combination of gellan and of at least one other gelling agent selected from the group consisting of gelatin and hydrocolloids such as agar, carragheenan, xanthan gum, alginate, dextran, curdlan, welan gum, rhamsan gum or modified starches.

According to a preferred embodiment of the invention the filler and the sequestering agent, are as described hereinabove.

According to another embodiment, the shell further comprises a plasticizer as described hereinabove and advantageously a coloring agent.

The amount of plasticizer ranges from 1% to 30% by weight, preferably from 2% to 15% by weight, and even more preferably from 3 to 10% by weight of the total dry weight of the shell.

According to the intended use of said capsules, the shell may contain other additives such as perfumes, aromas, etc.

According to a preferred embodiment, the breakable capsule according to the invention has a crush strength of from 0.01 to 5, preferably from 0.01 to 2.5 kp.

Advantageously, the shell thickness of the capsule is 10-500 microns, preferably 30-150 microns, more preferably 50-60 microns. The ratio diameter of the capsule/thickness of the shell is in the range of 10 to 100, preferably 50 to 70.

The core of the capsule is preferentially composed of a mixture of materials or products which are lipophilic or partially soluble in ethanol, or of molecules formulated as oil/water/oil emulsions.

The core of a breakable capsule according to the invention represents by weight 50 to 92% of said capsule, preferably 60 to 90%, more preferably 70 to 80%.

The core of the capsule may be composed of one or more lipophilic solvents conventionally used in the food, pharmaceutical or cosmetic industries. In a preferred embodiment, these lipophilic solvents may be triglycerides, especially medium chain triglycerides, and in particular triglycerides of caprylic and capric acid, or mixtures of triglycerides such as vegetable oil, olive oil, sunflower oil, corn oil, groundnut oil, grape seed oil, wheat germ oil, mineral oils and silicone oils. The amount of lipophilic solvent in the core of a capsule according to the invention is of the order of 0.01 to 90%, preferentially 25 to 75%, of the total weight of the capsule.

The core may also comprise one or more aromatic or fragrance molecules as conventionally used in the formulation of flavoring or fragrance compositions. Mention will in particular be made of aromatic, terpenic and/or sesquiterpenic hydrocarbons, and more particularly essential oils, alcohols, aldehydes, phenols, carboxylic acids in their various forms, aromatic acetals and ethers, nitrogenous heterocycles, ketones, sulfides, disulfides and mercaptans which may be aromatic or non aromatic. It may also comprise one or more molecules or extracts for cosmetic use.

The core may also comprise one or more fillers as used in aromatic emulsions. Mention will be made of dammar gum, wood resins of the ester gum type, sucrose acetate isobutyrate (SAIB) or brominated vegetable oils. The function of these weighting agents is to adjust the density of the liquid core.

The core may also comprise one or more sweeteners, which may be provided in the form of a solution or suspension in ethanol. Examples of suitable sweeteners may be, but is not limited to, aspartame, saccharine, NHDC, sucralose, acesulfame, neotame, etc.

The core may also comprise one or more “sensate” aromatic agents, which provide either a freshening effect or a hot effect in the mouth. Suitable freshening agents may be, but are not limited to, menthyl succinate and derivatives thereof, in particular Physcool® marketed by the Applicant. A suitable hot effect agent may be, but is not limited to, vanillyl ethyl ether.

The flavoring agents that can be solubilized in the solvent of the core of the capsule include, but are not limited to, natural or synthetic aromas and/or fragrances. Examples of suitable fragrances are fruity, confectionery, floral, sweet, woody fragrances. Examples of suitable aromas are vanilla, coffee, chocolate, cinnamon, mint.

The capsules according to the invention can be used in many applications such as food, pharmaceutical, cleaning and cosmetic products.

They can be presented and sold in a slurry containing them, in suspension in a gel formed with a gel forming agent such as CMC or Carbopol, and optionally comprising preservatives and stabilizers.

The invention is hereunder illustrated by the following examples, which should not be considered as limiting the scope of the invention.

### BRIEF DESCRIPTION OF THE DRAWING

FIG. 1 is a plot of wet capsule crush strength (gel strength) measured for both capsules A1a and A1b using a texturometer, comparing influence of concentration of calcium.

### DESCRIPTION OF THE PREFERRED EMBODIMENTS

### Examples

### Example 1

Menthol Capsules (referred as 3039/A1) are prepared by co-extruding an outer liquid phase and an internal liquid phase presenting the following compositions:

| Outer liquid phase | %/total | %/dry |
|---|---|---|
| Dry matter: 15.0% | weight | matter |
| gellan | 2.000% | 13.33% |
| Sorbitol | 1.000% | 6.67% |
| Dextrin Cristal Tex | 11.400% | 76.00% |
| 648 | | |
| Sodium citrate | 0.200% | 1.33% |
| Citric acid | 0.1% | 0.67 |
| unipure blue | 0.300% | 2.00% |
| pigment CI77007 | | |
| Deionized water | 85.000% | 100% |
| | 100.000% | |
| Internal liquid | | |
| phase | % | % |
| Ethanol | 5.0000% | |
| Miglyol 812S | 81.5000% | |
| Menthol codex | 13.5000% | |
| Total | 100.0000% | 100.00% |

The obtained capsules are separated into two batches referred as A1a and A1b. Capsules from each batch are cooled at 4° C. for 1 h, washed with desionised water and then immersed in a bath containing an aqueous solution of calcium chloride (0.1% for A1a and 1% for A1b) at pH=3.5 at T=20° C. during 15 minutes.

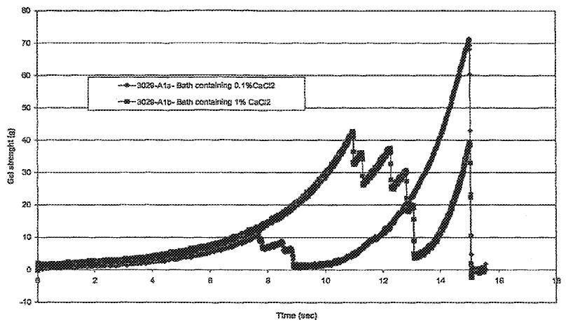

Wet capsule crush strength (gel strength) is then measured for both capsules A1a and A1b using a texturometer TA.XT plus from Micro Stable System to compare influence of concentration of calcium (the results are presented on FIG. 1).

Wet capsule strength is higher using 1% CaCl₂) solution than using 0.1% CaCl₂) solution.

After drying, crush strength of the capsules is measured using a texturometer in compression mode.

| | 3039/A1a | 3039/A1b |
|---|---|---|
| Crush strength | 184 g | 186.6 g |
| (dry capsules) | | |

The obtained capsules present the following physical characteristics:

diameter: 2 mm,

thickness of the shell: 0.096 mm,

total weight: 4 mg,

weight of the core: 2.8 mg (70%),

weight of the shell: 1.2 mg (30%).

### Example 2

Cinnamon Capsules (referenced as 4053/F1) are prepared by co-extruding an outer liquid phase and an internal liquid phase presenting the following compositions:

| Outer liquid phase | %/total | %/dry |
|---|---|---|
| Dry matter: 13.0% | weight | matter |
| gellan | 2.000% | 15.38% |
| Sorbitol | 1.000% | 14.62% |
| Dextrin Cristal Tex | 8.500% | 65.38% |
| 648 | | |
| Sodium citrate | 0.200% | 1.54% |
| Calcium citrate | 0.100% | 0.77% |
| Titanium dioxide | 0.300% | 2.31% |
| Osmosed water | 87.000% | 100% |
| | 100.000% | |
| Internal liquid | %/total | % without |
| phase | weight | ethanol |
| Ethanol | 5.0000% | |
| Miglyol 812S | 58.9000% | 85.79% |
| Cinnamon | 19.6000% | 14.21% |
| Physcool | 10.0000% | 10.53% |
| N-ethyl-p-menthane- | 6.5000% | 6.84% |
| 3-carboxamide | | |
| commercialy | | |
| available as WS3 | | |
| Total | 100.0000% | 100.00% |

The obtained capsules are cooled at 4° C. for 1 h, washed with desionised water and then immersed in a bath containing an aqueous solution containing 1.25% of calcium chloride at pH=3 at T=20° C. during 30 minutes.

The obtained capsules present the following physical characteristics:

diameter: 1.2 mm,

thickness of the shell: 0.053 mm,

total weight: 0.87 mg,

weight of the core: 0.62 mg (71.98%),

weight of the shell: 0.24 mg (28.02%),

Capsules are then incorporated into a toothpaste base containing mint flavour and cinnamon capsules 4053/F1 at a 0.2% use level. During brushing, cinnamon flavour is clearly identified showing good breakability of the capsules.

1-26. (canceled)

27. A seamless breakable capsule comprising:
an oily core comprising one or more aromatic or fragrance molecules; and
a shell composition surrounding the oily core, the shell composition comprising:
a gelling agent comprising gellan gum alone or in combination with another gelling agent,
a filler selected from the group consisting of starch derivatives such as dextrin, maltodextrin, alpha cyclodextrin, beta cyclodextrin, and gamma cyclodextrin, or, cellulose derivatives, such as hydroxypropylmethylcellulose (HPMC), hydroxypropylcellulose (HPC), and methylcellulose (MC), and mixtures thereof, the filler being from 25 to 95% by weight on the total dry weight of the shell composition, and
a divalent metal sequestering agent selected from the group consisting of trisodium citrate, trisodium phosphate, tetrasodium pyrophosphate, sodium hexametaphosphate and mixtures thereof, the amount of sequestering agent being at most 2% by weight on the total dry weight of the shell composition,
wherein said capsule is incorporated into a fluid medium and has a shell composition which is water impermeable
wherein said and has a crush strength between 0.1 and 2.5 kp and
wherein the filler allows increasing the dry material amount of the capsule shell.

28. The seamless breakable capsule according to claim 27, wherein the shell composition further comprises a plasticizer selected from the group consisting of glycerol, sorbitol, maltitol, triacetine, and mixtures thereof, the plasticizer being from 0.1 to 30% by weight of the total dry weight of the shell composition.

29. The seamless breakable capsule according to claim 27, wherein the gelling agent of the shell composition is a combination of gellan and one gelling agent selected from the group consisting of gelatin, agar, carrageenan, pectins, xanthan gum, cellulose gum, alginate, dextran, curdlan, welan gum, rhamsan gum and modified starches.

30. The seamless breakable capsule according to claim 27, wherein the gelling agent of the shell composition is 4 to 95% by weight of the total dry weight of the shell composition.

31. The seamless breakable capsule according to claim 27, wherein the gelling agent is gellan gum alone.

32. The seamless breakable capsule according to claim 27, wherein, when the gelling agent of the shell composition is used in combination with at least another gelling agent, the weight ratio between gellan gum and the other gelling agents is from 80/20 to 20/80.

33. The seamless breakable capsule according to claim 27, wherein the amount of the filler of the shell composition is from 50 to 80% by weight on the total dry weight of the shell composition.

34. The seamless breakable capsule according to claim 27, wherein the amount of sequestering agent of the shell composition is at most 0.5% by weight of the total dry weight of the shell composition.

35. The seamless breakable capsule according to claim 27, wherein the shell composition further comprises an acid salt selected from the group consisting of citrate, glucuronate, adipate, fumarate, gluconate and salt of glucono-delta-lactone, and mixtures thereof.

36. The seamless breakable capsule according to claim 27, wherein the shell composition further comprises a plasticizer, the plasticizer being from 0.1 to 30% by weight of the total dry weight of the shell composition.

37. A slurry containing co-extruded and breakable capsules according to claim 27, in suspension in a gel formed with a gel forming agent selected from the group consisting of CMC, xanthan gum, and Carbopol, and optionally comprising preservatives and stabilizers.

38. A food product including breakable capsules according to claim 27.

39. An oral care product including breakable capsules according to claim 27.

40. A pharmaceutical product including breakable capsules according to claim 27.

41. A fragrance including breakable capsules according to claim 27.
